(19) United States
(12) Patent Application Publication
(10) Pub. No.: US 2004/0247534 A1
(43) Pub. Date: Dec. 9, 2004

(54) Foamable fluoride gel compositions and methods of treatment using the same

(75) Inventor: Edwin I. Stoltz, Delray Beach, FL (US)

Correspondence Address: STEINBERG & RASKIN, P.C., 1140 AVENUE OF THE AMERICAS, 15th FLOOR, NEW YORK, NY, 10036-5803, US

(73) Assignee: Sultan Dental Products, Ltd

(21) Appl. No.: 10/638,986

(22) Filed: Aug. 12, 2003

Related U.S. Application Data
(60) Provisional application No. 60/476,411, filed on Jun. 6, 2003.

(51) Int. Cl. A61K 7/18; A61C 5/00

(52) U.S. Cl. 424/52; 433/217.1

(57) ABSTRACT

Compositions and methods of treating teeth with self-foaming fluoride-containing gels are disclosed. In preferred aspects, the method includes dispensing a self-foaming fluoride-containing gel from an aerosol container into the trough of a dental tray, placing the trough of the dental tray containing the self-foaming fluoride containing gel into engagement with a patient's teeth and allowing the gel to transform into a foam while the dental tray is in engagement with the teeth. The fluoride-containing gels include a water soluble fluoride component, an oil-in-water emulsifier, a micro-emulsion stabilizer, a surfactant, a micro-emulsion stabilizer and thickener and optionally a gel clarifying agent.

### CROSS-REFERENCE TO RELATED APPLICATIONS

This application claims the benefit of priority from U.S. Provisional Patent Application Ser. No. 60/476,411, filed Jun. 6, 2003, the contents of which are incorporated herein by reference.

### BACKGROUND OF THE INVENTION

1. Field of the Invention

The present invention is directed to improvements in methods of treating teeth with fluoride. In particular, the invention is directed to durable and stable dental fluoride foam compositions made from self-foaming gels.

2. Brief Description of the Prior Art

The use of fluoride to treat dental plaque is well documented. For example, fluoride is often added to community potable water supplies, consumer products such as rinses, gels, foams, and of course toothpastes in order to reduce dental caries.

Over the years, various fluoride compounds such as sodium fluoride, stannous fluoride or sodium monofluorophosphate have been used to provide the beneficial activity required to reduce, inhibit, control and prevent dental plaque, and consequently dental cavities and decalcification of tooth enamel. Professional dental practitioners often use fluoride gels and foams to affect a high degree of plaque control and prevention.

For optimum results, fluoride gels are applied by the dental professionals using dental trays which fit over the upper or lower teeth at the same time and allows the gel to directly contact the teeth for periods of one to four minutes and optimize fluoride uptake by the tooth enamel. While these products are called “gels”, they are really viscous liquid products that are poured into the dental tray. Although dental trays tend to be more effective than toothbrushes, currently available fluoride gels lack sufficient stability to remain in the oral cavity for the time required for maximum therapeutic effect. The problem is especially critical when the dental tray is turned upside down to submerge the lower teeth in the fluoride containing gel. In these situations, the gel quickly leaves the dental tray and reduces the effectiveness of the fluoride treatment.

One attempt to address the shortcomings of currently available products is set forth in U.S. Pat. No. 4,770,634. This patent discloses an aerosol, foamable fluoride product which can be dispensed into the trough of a dental tray. The patentees describe the foam as being dense, stable and non-flowable. The compositions prepared in accordance with the '634 patent, however, also demonstrate the aforementioned physical stability problems, especially when used in dental trays which are turned upside down to treat the lower teeth.

One of the chief drawbacks associated with these fluoride foams is that they are prepared using substantially all water-soluble ingredients. On one hand, water soluble and hydrophilic ingredients make preparing fluoride-based foams easy to formulate, which produce sufficient amounts of foam. On the other hand, the physical nature of such foam ingredients dictates that the foams and foam cell structure will rapidly dissipate in the presence of (aqueous) saliva due to the inherent weak stability of a hydrophilic foam under acid conditions and will fail to remain in intimate contact with the teeth for one to four minutes.

Another shortcoming associated with prior art dental foams is the fact that they must be dispensed from their pressurized containers at angles of about 90 degrees into the fluoride dental trays. This results in large amounts of the product left in the container after the propellent has been dissipated. A more economical solution is therefore sought to this problem.

In view of the foregoing, there is still a need for improved dental fluoride compositions, especially in the gel foam type formulations. The present invention addresses this need.

### SUMMARY OF THE INVENTION

It is an object of the present invention to provide improved, true clear fluoride gels that turn into dense, stable foams within about 10 to about 60 seconds.

It is a further object of the present invention to provide foamable dental fluoride compositions which demonstrate enhanced foam stability in the oral cavity.

A still further object of the present invention is to provide improvements in treating teeth with fluoride-based compositions.

Another object of the present invention is to provide a method of preparing gel foamable dental fluoride compositions, which demonstrate enhanced foam stability in the oral cavity for time periods of from about one to about four minutes.

These objects as well as others are achieved by the present invention which, in one aspect, includes a foamable dental gel fluoride composition containing a water soluble fluoride component present in an amount sufficient to provide from about 0.5 to about 10% by weight available fluoride, from about 2.0 to about 30% by weight of an oil-in water emulsifier, from about 0.5 to about 30% by weight of a surfactant, from about 0.5 to 5.0% by weight of a micro-emulsion thickener, and, optionally, from 0.5 to 5% by weight of a gel clarifying agent. These ingredients are made into a micro-emulsion which is then combined in an aerosol container with a suitable propellant such as for example isopentane, n-butane, isobutane, propane and mixtures thereof. Preferred aerosol containers are those known in the art as bag-in-can systems wherein the barrier bag contains the micro-emulsion (gel) and from about 2.0 to about 6.0% by weight and preferably about 3% by weight of a (first) propellant having a vapor pressure of from about 10 to about 46, and preferably from about 7.0 to about 31.0 psig. In certain preferred aspects of the invention, the first propellant within the barrier bag is actually a blend of two or more propellants wherein one propellant has a low to negative vapor pressure. The aerosol can includes a second propellant surrounding the barrier bag within the can having a vapor pressure which is preferably from about 14 to about 20 psig and preferably at least about 16.0 psig greater than the barrier bag propellant. The second propellant is present in amounts of from about 2.0 to about 8.0% by wt.

The foamable dental fluoride gel compositions of the present invention are micro-emulsions which contain a combination of hydrophobic and hydrophilic ingredients. This combination makes the fluoride gel-based foam more durable and demonstrates increased micro cell stability in the oral cavity.

In another embodiment of the invention there are provided methods of treating teeth with fluoride. The methods include dispensing a self-foaming fluoride-containing gel such as that described above from an aerosol container into the trough of a dental tray, placing the trough of the dental tray containing the self-foaming fluoride containing gel into engagement with the teeth to be treated, and thereafter, allowing the self-foaming gel to transform into a foam while said dental tray is in engagement with the teeth.

In this aspect of the invention, the pressurized and foamable fluoride containing composition, which is an oil micro-emulsion, is dispensed from an aerosol container to form a preferably clear gel that becomes a foam, preferably within about a minute or less. In preferred embodiments, the fluoride-containing gel is placed in contact with the teeth prior to the gel completely transforming into the micro-cell foam so that the transition from gel to foam occurs while the gel is in contact with the teeth. The methods of treatment takes advantage of the increased oral cavity stability afforded by the foams described herein to provide enhanced fluoride uptake by the tooth enamel. The contacting of the teeth with the foam can be in either an acidulated or a neutral medium to effect fluoride uptake by the teeth.

Further aspects of the present invention include methods of preparing the micro-emulsion and methods of preparing aerosol containers containing the foamable fluoride dental gel compositions described above.

As a result of the present invention, there are provided stable foamable dental fluoride products which demonstrate significantly greater durability after being dispensed into dental trays and, more importantly, after the self-foaming gel has been placed in the oral cavity where contact with patient saliva is unavoidable. Furthermore, the durable nature of the resultant fluoride foam of the present invention allows the practitioner to be assured that a sufficient amount of the fluoride is present to intimately act upon the dental enamel and allow therapeutic fluoride ion uptake.

Further advantages of the methods of treatment described herein include the fact that unlike the prior art fluoride foams, the aerosol gel compositions of the present invention can be applied to the tooth surface as a gel or nearly gel-like material which becomes a post-foaming fluoride-containing foam thereafter (i.e. once it is in contact with the teeth) to maximize the therapeutic effect of the treatment. While applicant is not bound by theory, it is believed that, in certain preferred embodiments, specific propellant-microemulsion mixtures, in combination with micro-emulsion stabilizers and thickeners such as Crothix, actually delay the immediate formation of foam exiting the pressurized can. This allows the foam formation to essentially initiate at the same time and/or after it is placed in contact with the teeth. This post-foaming method of treatment allows maximum amounts of fluoride to be delivered to the patient.

A further advantage of the present invention includes the fact that there are provided true clear gel fluoride compositions for treating teeth. The inventive gels can be dispensed into suitable dental trays and, within about a minute, the gel expands and develops into a stable micro-cell foam which encompasses tooth surfaces more effectively than the fluoride foams of the prior art. For example, because of the delay in the transformation of the gel into a foam after dispensing, the artisan has the time to position the dental tray (and gel therein) into the patient's mouth and allow the expansion of the gel into the micro-cell foam structure in situ. The “explosion” of the gel into the expanding micro-cells actively causes more of the solubilized fluoride to expand into areas where there are tooth crevices in the oral cavity which were often insufficiently treated with prior art foams. Thus, an improved method of treatment and/or preventing dental cavities is provided as compared to foams which do not expand in the oral cavity but instead tend to rapidly dissipate.

For purposes of the present invention, the term “orally compatible” shall be understood to describe compositions and ingredients which are generally regarded as safe for use in the oral cavity. Additionally, for purposes of the present invention, the terms “self-foaming” and “post-foaming” shall be understood to describe the quality of the micro-emulsion based gels to briefly remain gels after being dispensed under pressure from an aerosol container and, after a predetermined time period, begin to transform into a foam without significant external agitation, heat, etc.

For a better understanding of the present invention, together with other and further objects, reference is made to the following and the scope of the present invention will be pointed out in the appended claims.

### DETAILED DESCRIPTION OF THE INVENTION

The foamable dental fluoride gel compositions of the present invention include an oil-in-water micro-emulsion containing:

a) a water soluble fluoride component present in an amount sufficient to provide from about 0.5 to about 10.0% by weight available fluoride;

b) from about 2.0 to about 30.0% by weight of an oil-in-water emulsifier;

c) from about 0.5 to about 30.0% by weight of a surfactant;

d) from about 0.5 to about 5.0% by weight of a micro-emulsion thickener;

and optionally

e) from about 0.5 to about 10.0% by weight of a gel clarifying agent.

The remainder of the gel composition includes up to about 65-70% water and less than about 5% inactives such as sweeteners, colorants, etc. In preferred aspects of the invention, the water soluble fluoride component provides from about 0.5% to about 5% available fluoride. Most preferably, the water soluble fluoride component provides from about 1% to about 3% available fluoride. The water soluble fluoride component can be selected from materials well known to those of ordinary skill. Without being limited thereto, examples of suitable fluoride sources include sodium fluoride, sodium monofluoro-phosphate, stannous fluoride and the like. Additional choices include fluoroalkylphosphate salts such as monoammonium 1,1,7-trihydroperfluoroheptyl phosphate, described in U.S. Pat. No. 2,955,985 and/or quaternary ammonium fluorides, such as doceyltrimethylammonium fluoride, described in U.S. Pat. No. 3,124,512. The disclosure of each of the foregoing patents is incorporated by reference herein. Mixtures of the foregoing fluorides such as a combination of sodium fluoride and one or more of the aforementioned ingredients are also contemplated. Other orally compatible water soluble fluorides containing compositions not specifically mentioned, but known to those skilled in the ordinary art can also be included herein.

Sodium fluoride is particularly well suited for use in the compositions of the present invention and can be present in the formulation in amounts ranging from about 1.0% to about 22.2% by weight, so as to allow the gel foam to deliver the above-mentioned range of available fluoride. Preferably, the sodium fluoride will comprise from about 1.0% to 10% by weight, and most preferably from about 2% to 6% by weight. Those of ordinary skill in the art will of course realize that the actual amount of water soluble fluoride component in the composition will be greater than the amount of fluoride delivered and will vary the amount according to the type of fluoride component used. The gel foam compositions of the present invention are based on the amount of available fluoride delivered by the component rather than by the weight of the ingredients. The amounts of specific water soluble fluoride ingredients required to deliver the desired fluoride amounts will be apparent to those of ordinary skill without undue experimentation.

One of the keys to the foams of the invention is their durability in the oral cavity. The gel foams are based on oil-in-water (hydrophobic) micro-emulsions, rather than an aqueous or hydrophilic systems which rapidly dissipate in the oral cavity. A micro-emulsion is a dispersed system containing at least two immiscible liquid phases. In order for a micro-emulsion to be stable, it must contain at least three (3) components, i.e., the dispersed phase, the dispersion medium, and the emulsifying agent. Preferably, micro-emulsions formed in accordance with the present invention are oil-in-water micro-emulsions with the dispersed phase being an oil, dispersed as droplets throughout the aqueous dispersion medium. Suitable oil-in-water emulsifiers or emulsifying agents are discussed below.

As mentioned above, the fluoride gels of the present invention include from about 2.0 to about 30% by wt. of an oil-in-water emulsifier. In certain preferred aspects of this embodiment, the emulsifier is a blend of at least two separate emulsifiers which, for purposes of the present invention are designated as first and second emulsifiers. The ratio of the first emulsifier to said second emulsifier will vary according to the needs of the artisan but is preferably about 2:1. More specifically, the first emulsifier comprises from about 1.0 to about 20.0% and preferably from about 2.0 to about 6.0% by weight of the gel. The second emulsifier, which is selected based on its ability to provide a stabilizing effect on the first emulsifier, is present in amounts of from about 1.0 to about 10.0% by weight, with amounts of from about 1.0 to about 6.0% by weight being preferred.

Suitable oil-in-water emulsifiers can be selected from among those orally compatible emulsifying agents well known to those of ordinary skill. In some preferred aspects, the oil-in-water emulsifier contributes to initiating and maintaining the desired acid pH range of the gel foam. An illustrative and non-limiting list of suitable acidic oil-in-water emulsifiers include cetyl phosphate, stearic acid, PPG-5, ceteth phosphate, cetearyl alcohol, dicetyl phosphate, ceteth-10 phosphate, polyoxyethylene (10) oleyl alcohol phosphate, hereinafter known as “oleth-10-phosphate” which is available from Croda Inc. as Crodafos N10 Acid, polyoxyethylene (3) oleyl alcohol phosphate known as “oleth-3-phosphate” (as taught in U.S. Pat. No. 5,824,289) also available from Croda, Inc., as Crodafos N3 Acid, cetostearyl alcohol, stearyl alcohol, oleyl alcohol, behenic acid, cetyl phosphate, stearic acid, and related fatty alcohols with linear carbon chains and wax-like materials having high molecular weights such as phosphated cetyl ether, i.e., Crodafos CAP acid. Mixtures of the foregoing are also contemplated. Preferably, however, in some aspects of the invention where a blend of emulsifiers are used, the first emulsifier is oleth-10-phosphate and the second emulsifier is oleth-3-phosphate. In these preferred aspects of the invention, the first emulsifier comprises about 2-4% by weight and the second emulsifier comprises about 1-2% by weight respectively of the inventive fluoride gel compositions. Such combinations provide dental gels capable of resolving by itself into a dental foam compositions having a pH from about 2.5 to about 3.5.

The dental gel compositions of the present invention can also include a surfactant that contributes to the emulsification of the hydrophobic emulsifier and emulsion stabilizer present in the composition of the foamable dental fluoride gel. The surfactant is preferably water soluble and provides a dual function in the aerosol fluoride gel foam composition. First, the surfactants help to lower the surface tension in coupling all of the ingredients to form a clear gel. Secondly, the surfactants assist in the forming of a dense, micro-cell fluoride foam for dental treatment, when combined with the aerosol propellants. As mentioned above, the surfactant is present in amounts of from about 0.5 to about 30.0% by weight, based on its purity being 100%. Preferably, the surfactant is present in amounts ranging from about 0.5 to about 10% by weight. A non-limiting list of surfactants includes those which are anionic, nonionic and amphoteric. The cationic surfactant class, however, was found to provide limited use in the fluoride dental gels of the present invention. Therefore, one skilled in the art can choose surfactants from each of the surfactant type classes. Illustrative and not limiting examples of the three (3) major classes of surfactants are described below:

“Anionic” surfactants can include: sodium lauryl ether sulfate, ammonium lauryl ether sulfate, sodium lauryl sarcosinate, sodium N-methyl-N-oleyl-taurate and disodiumoleyl sulfosuccinate, sodium N-methyl cocyl taurate, etc.

“Nonionic” surfactants can include: Nonylphenolethoxylate, Myristyl-cetyl dimethyl oxide, lauric acid diethanolamine, polyoxyethylene propylene glycol stearate nonylphenoxypoly(ethyleneoxy) ethane, known in the trade as “Rhodapex CO-630”, octylphenoxypoly(ethyleneoxy) ethanol, known in the trade as “Rhodapex CA-630”, both marketed by Rhodia Inc., Parsippany, N.J.

“Amphoteric” surfactants can include: cocoamidopropyl betaine, lauryl amine oxide, cocoimidazoline monocarboxylate, cocoarnphocarboxyl glycinate, etc.

“Cationic” surfactants can include: bis (2-hydroxy-ethyl) cocoaoxide, cetyl dimethyl benzyl ammonium chloride, dimethyl-aminopropylamine, etc.

Although many surfactants can be employed, it is important to pick those which have good foaming properties. As pointed out above, a key feature of the fluoride gel compositions of the invention is their ability to foam by itself after being dispensed from an aerosol can and remain stable throughout the dental treatment process. It is widely understood in the dental profession that therapeutic fluoride foam must be capable of retaining its stable form for up to about four (4) minutes in the patient's mouth. The surfactant also assists the propellant in forming the stable micro-cell foam structure with greater surface area that results in a stable dental foam. It will be appreciated that a wide variety of orally compatible anionic, nonionic, amphoteric and in some cases cationic surfactants can be used in the compositions of the present invention. Preferably however, the surfactants of choice for the present invention are nonylphenoxypoly(ethyleneoxy) ethane “Rhodapex CO-630” and octylphenoxypoly (ethyleneoxy) ethanol “Rhodapex CA-630”. Sodium lauryl sarcosinate as a 30% active anionic surfactant, available under the trademark “Hamposyl L-30” by Dow Chemical Co., Midland, Mich. is also a preferable surfactant as are the amphoteric surfactants N-lauryl myristyl beta aminopropionic acid, disodium N-lauryl beta-iminidipropionate. Mixtures of the surfactants are also contemplated.

The foamable gel dental fluoride compositions of the present invention can also include a micro-emulsion thickener which may also confer an emulsion stabilizing effect. A non-limiting list of orally compatible thickeners includes materials such as cetyl alcohol, also known as 1-hexadecanol, available from several manufacturers, sodium monostearate and PEG-150 pentaerythrityl tetrastearate, known in the trade as Crothix. Preferably, the micro-emulsion thickener is Crothix. As mentioned above, the thickener is present in amounts of from about 0.5 to about 5% by weight. Preferably, however, it is present in amount from about 1.0 to 3.0% by weight. Mixtures of micro-emulsion thickeners are also contemplated.

In some preferred aspects of the invention, a clarifying agent is included to insure the clarity of the gel. The clarifying agent can be present in amounts of from about 0.5 to 10% by weight and preferably in amounts of from 1.0 to 6% by weight. One preferred chemical clarifier for the inventive compositions is specially denatured ethyl alcohol with a purity of 200 proof. The denaturant of choice is “benzaldehyde” at a concentration of 1.5% by weight. This denaturant has an almond taste that is compatible with various flavors, such as cherry, bubble-gum or orange. Those familiar with the art could also use 190 or 200 proof specially denatured ethyl alcohol (SDA) using the oils of peppermint, spearmint or wintergreen as denaturants that are readily available from commercial suppliers such as Aaper Alcohol & Chemical Co., Shelbyville, Ky.

The fluoride gel compositions of the present invention can also include one or more ancillary ingredients to provide commercially acceptable products. Such ingredients will be apparent to those of ordinary skill in the art and include sweetening agents such as sodium saccharin, aspartame, sorbitol, preservatives such as sodium benzoate or potassium sorbate, flavorants, colorants, etc. The amounts of such ingredients included in the gels will vary somewhat due to the specific ingredient selected and the needs of the artisan. It is contemplated that the amounts will be apparent to those of ordinary skill but nonetheless range from about 0.001 to 10% by weight.

In accordance with another key aspect of the invention, there is provided a method of treating teeth with fluoride. The method includes:

a) dispensing a self-foaming fluoride-containing gel from an aerosol container into the trough of a dental tray;

b) placing the trough of the dental tray containing the self-foaming fluoride containing gel into engagement with the teeth to be treated, and

c) thereafter, allowing the self-foaming gel to transform into a foam while the dental tray is in engagement with the teeth and thereby facilitate fluoride treatment of the teeth.

The durable nature of the fluoride foam which is generated in the oral cavity during treatment allows the practitioner maximize the amount of the fluoride intimately acting upon the dental enamel, and thus maximizing therapeutic fluoride ion uptake. The post-foaming (after dispensing from the aerosol container) of the fluoride-containing gel while in contact with the teeth overcomes a significant drawback of the prior art compositions which quickly dissipated after insertion in the oral cavity.

In use, an aerosol container having a dispensing spout is rotated to align the dispensing spout with the trough of a dental tray (the size, shape and other dimensions being well known to those of ordinary skill). The actuator is pressed to dispense a sufficient amount of the gel-based compositions of the invention into the trough. For purposes of the present invention, “sufficient amount” shall be understood to mean an amount which provides a therapeutic amount of fluoride to the patient being treated. The amount of gel dispensed into the trough will generally be somewhat less than the practitioner dispenses with prior art foams which are typically used in amounts that substantially fill the volume defined by the trough. Instead, the gel-based compositions of the present invention can be dispensed as a bead or ribbon-like shape along the length of the trough into approximately the center portion thereof so that when the post-foaming of the gel occurs, preferably while in the oral cavity of the patient being treated), there is still room in the trough to contain the foam not contacting the teeth. The gel-containing tray is then placed in a patient's mouth so as to superimpose the trough and its fluoride-containing composition about and into engagement with the teeth to be treated. The post-forming fluoride foam is maintained in engagement with the teeth for about 1 to 4 minutes to effect the fluoride treatment of the teeth.

The foam that is formed in the trough is a stable microcell which resists rapid dissipation after contact with the saliva present in the oral cavity by virtue of its hydrophobic rather than hydrophilic characteristics. In spite of the stable nature of the fluoride foams, the residual amounts which remain on the tooth surfaces after the treatment period is over are nonetheless quickly and easily removed by simple aspiration or water rinsing of the mouth.

In another aspect of the invention, there are provided methods of preparing the gel concentrate that readily becomes a dental foam upon being dispensed into a dental tray. The methods include:

a—preparing an oil-in-water micro-emulsion concentrate containing a water soluble fluoride component in an amount sufficient to provide from about 0.5% to about 10% by weight available fluoride; and

b—combining the gel micro-emulsion concentrate with aerosol propellant(s) to form a preferably clear stable gel that will foam in less than about a minute after dispensing to become a foaming gel dental fluoride composition. In practice, the oil-in-water micro-emulsion will preferably contain not only the water soluble fluoride, but also each of the ingredients in the amounts mentioned above, i.e., oil-in-water emulsifier, micro-emulsion stabilizer, surfactant, micro-emulsion thickener; and optionally a gel clarifying agent as well as any ancillary ingredients such as flavorants, sweeteners, etc.

As noted above several times, the gel dental fluoride foam uses oil-in-water emulsifying agents that are not easily made soluble in water. Therefore, in those aspects of the present invention where a clear gel concentrate that does not have any undissolved particulate matter is desired, it is necessary to provide relatively high heat combined with a shearing type mixer for complete dissolution. It is therefore preferable to maintain the batch temperatures listed below, in order to produce clear gel concentrates. Where such clarity is not required, the ranges mentioned below can be varied or lowered somewhat in accordance with generally acceptable procedures.

The compositions of the present invention can be prepared in the following general manner. Those of ordinary skill in the art will realize that modifications can be made to the illustrative procedure without departing from the steps necessary for forming a dental gel foaming fluoride composition. A typical oil-in-water emulsion compounding procedure includes the following compound steps:

Step #1. The prescribed quantity of water usually about 55 to about 65% by weight of the formula (which can be ultra-violet lamp treated deionized water or other sterilized water) is charged into a sanitized stainless, scale mounted batch tank that is equipped with an internal heat source and power mixer. Heating is started immediately, but mixing is not started until the water level is over the mixer propellers or other mixer head. Complete the water filling process and record fill weight. Stop and maintain heat at about 185-190° F.

Step #2. The liquid foam stabilizer such as glycerin is added, followed by a sweetening agent, e.g., sorbitol, with continued heating and mixing.

Step #3. The water temperature is allowed to rise back to about 185-190° F. or a temperature which sufficient to get the oil-in-water emulsifier, e.g., oleth-10-phosphate, into solution. In fact, the oil-in-water emulsifier can be added while temperature is rising to the target temperature desired, e.g. 190° F., with continued mixing. Once the oil-in-water emulsifier is in solution, the other ingredients can be added.

Step #4: Once the batch temperature has reached 185-190° F., the “micro-emulsion stabilizer” acidulated is added to the batch, e.g., oleth-3-phosphate. After being assured that the oil-in-water ingredients, e.g. the oil-in-water emulsifier and micro-emulsion stabilizer are completely in solution, the heat source is turned off and the surfactant is added.

Step #5: Mixing of the heated batch is continued while trying to minimize the creation of any excess foam. The remaining ingredients are not added to the batch until it cools down to about 160-170° F. At this point the micro-emulsion gel thickener is added, e.g., PEG-150 Penta, known in the trade as Crothix. With continued mixing the compounder will see that the batch is getting thick and will require some additional mixing speed.

Step #6: Cooling of the batch is allowed to continue and when it reaches a temperature of about 125-130° F., the gel clarifier, if included, is added, e.g., SD Alcohol 38B, 200 proof.

Step #7: The sodium fluoride followed by the ancillary or inactive ingredients are then added to the mixing batch and the fluoride content is confirmed before the batch is moved to the can filling line for aerosol production. The gel concentrate should have slow sustained mixing throughout the filling procedure at a holding temperature of 85-95 F.

The aerosol propellants required for the successful composition of the present invention are of prime importance. First, it is important to use propellants that are non-ozone depleting of the environment. Second, it has been determined that the well known hydrocarbon propellants, such as isopentane, n-butane, isobutane and propane, and mixtures thereof are the ideal sources of pressurization. The aerosol industry has designated these propellants with a numerical value to express the propellants vapor pressure, measured in pounds per square inch gauge, (psig).

We also found that the correct blend of propellants in the present invention are very important for a successful product. For example, isopentane whose vapor pressure is limited therefore must be assisted by either A-17 (n-butane) or A-31 (isobutane) to create the desired stable foam generation required for therapeutic fluoride dental treatment. The desired blend of isopentane and n-butane or isobutane are included and part of micro-emulsion gel concentrate within laminated bag in-can systems described below. The vapor pressure of the propellant in the bag ranges from about 10 to about 46 psig and is preferably from about 17 to about 31 psig. In most aspects of the invention, the propellant will be a mixture of two or more hydrocarbon propellants. Furthermore, the propellant included within the barrier bag will have a vapor pressure of at least about 17 psig, preferably at least about 14 to 20 psig less than the propellant(s) used in the can surrounding the barrier bag. Some preferred combinations include about equal amounts of A-17 (n-butane) and A-31 (isopentane), and about equal amounts of isopentane and A-17.

Thus, if isopentane and n-butane (A-17) are used in the concentrate (gel) within the bag, then a higher pressure propellant outside of the bag is required to exert its pressure on the bag-in-can. Likewise, if for example a blend of isopentane and isobutane (A-31) is used within the gel concentrate, we must use A-46 or even A-60 that is a blend of isobutane/propane (69%/31% by weight) outside of the bag-in-can. It is preferred that there be more pressure exerted upon the outer bag wall than the pressure within the laminated bag.

In certain preferred aspects of the invention, the inventive fluoride gels are dispensed from aerosol containers using as bag-in-can systems. The foregoing propellants are set forth for purposes of illustration and not exclusion. Those persons skilled in the art will realize that other propellants alone or in combination with others, can produce acceptable vapor pressures to produce an ideal gel fluoride foam and can also be included herein. An example of another potential propellant is HCPC-142b, chemically known as 1, 1, 1-chlorodifluorethane, with a vapor pressure of 29 psig.

In certain preferred aspects of the invention, the inventive fluoride gels are dispensed from aerosol containers using as bag-in-can systems. To produce the dental gel foamer of the present invention, one must use different aerosol components than those typically used in the art. One of the major components required for the formation of the aerosol gel is a special, rolled laminated bag known in the trade as the “ABS” barrier bag such as those made and distributed worldwide by CCL Inc., Penetanguishene, Ontario, Canada.

The barrier bag keeps the micro-emulsion gels and its propellants separate or apart from the propellant between the bag and inner can wall. This external can pressure helps to continually exert its higher pressure on the bag resulting in optimum gel being dispensed. A general process to produce the aerosol gels of the present invention includes the following steps:

Step #1. The barrier bag is either manually or automatically inserted into the empty aerosol can. It should be noted that the actual aerosol valve can be crimped or sealed to the laminated barrier bag hermetically.

Step #2. With the bag in place within the can, the unit moves to a gasser-crimper. The pressurizing at this point can easily be done, as known in the trade as “under the cap” gassing. The gassing of the can interior can be charged with a hydrocarbon propellant as shown in the present invention or compressed air. As the can is pressurized, the gasser-crimper automatically crimps the aerosol valve cup to the can for a hermetically perfect seal.

Step #3. The pressurized container then moves to the can filling station. At this time, the pressurized can and its empty bag enters a pressure filling machine. As the top of the can engages the pressure filler, the aerosol valve stem is depressed opening the valve to allow the pre-batched warm micro-emulsion gel with its propellants of choice, to be injected by force while under about 600 psi pressure. The high pressure forces the correct amount of gel concentrate into the barrier bag. The ratio of gel concentrate to propellant can range from about 90 to about 98% weight, with amounts of from about 96 to about 98% by weight being preferred. The forced injection of the gel and propellant concentrate now causes the rolled up laminated barrier bag to open up or fill out inside the can.

Step #4. After the container has been filled as described above, the can moves to the water bath with its moving conveyor. The charged pressurized can is held in the hot water until the internal temperature of the can reaches 130° F., as required by the U.S. Dot regulations. Any units showing propellant leaking are removed from the production lot.

Step #5. As the non-leaking can emerges from the hot water bath, an air jet blows the moisture from the valve cup. The unit moves on to have its actuator placed on the valve stem followed by the capping with its over closure.

While the aerosol production of the present invention is described for illustrative purposes above, there are other aerosol containers and methods of pressurizing that can be used for the present invention. Those of ordinary skill in the art will realize that other containers and methods can be used. A brief summary of the alternate aerosol components and processes is included here.

The “Sepro” can is another bag-in-can type of container. Here the micro-emulsion gel and propellant blend are added to the bag. The top of the bag is sealed onto a 1″ aerosol valve. The Sepro can has a small hole in its bottom through which a higher propellant is injected to encircle the outside of the filled bag. As the propellant is injected into the can through a partially open rubber plug, the plug is forced into the hole for a complete seal. Originally, the Sepro can was developed to keep the external propellant from the inner bag contents. Especially when there was a problem of compatibility. The pressure of the external higher pressure propellant continually exerts its pressure on the bag helping to easily dispense the gel product. However, for the present invention, we utilize the propellant blend within the gel micro-emulsion's bag to produce within seconds after being dispensed inside a dental tray to form a stable dental therapeutic fluoride foam.

There are other bag-in-can aerosol containers available to those skilled in the art that can be used to dispense gel products. The “Bi-Can” (short for bag-in-can) developed by the MB Group, plc, England, now part of CMB Packaging Ltd., There is also “Compack” development by A.S.M., S.S., which is similar to the “Lechner” system. This system uses a vertical pleated LDPE bag in an aluminum can with a 3.5 mm hole in the center of the can bottom. It should be noted here, that there are numerous “piston” type aerosol units available on the market today.

As with aerosol containers, it should be noted here that there exists in the trade alternative laminated bags available to those knowledgeable with aerosol technology. One example is the “Conally” bag that is constructed of low density polyethylene plastic, nylon and proprietary binding agents. The Lamicon bag is constructed also of LDPE and also ethyl acetate polymer with a special adhesive.

There are other ways to create gel products. For the present invention, however, it is preferable to use oil-in-water emulsifiers in the system. It was believed that the creation of a micro-emulsion gel was best suited to transform itself into a stable hydrophobic dental fluoride foam. It was also considered that as the dental gel-foam developed in the dental tray and quickly inserted into the patients mouth, that the acidulated fluorine ion would be able to penetrate each opening between and around the tooth enamel for complete coverage.

As noted above, the correct blend of propellants in the present invention is of prime importance. For example, isopentane, with a vapor pressure of −3 psig and therefore having limited post foaming properties, must be assisted by either A-17 (n-butane) or A-31 (isobutane). Thus, the desired blend and concentration of isopentane, n-butane or isobutane are preferably made a part of the micro-emulsion gel concentrate within the laminated bag-in-can.

### EXAMPLES

The following examples serve to provide further appreciation of the present invention, but are not meant in any way to restrict the effective scope of the invention.

### Example #1

The gel dental fluoride foam formulations of the Examples were made following the general procedure set forth below. In all examples, the sodium fluoride concentration was adjusted to reflect a final fluoride concentration of 1.23% wt., (±10%). The ingredients listed as “inactive” include the sweetening, preservation, flavor and coloring ingredients, these ingredients have no bearing on either the formation of the micro-emulsion gel concentrate or the stability of the resulting dental foam. The sum percentage by weight of all the ingredients of these inactive compounds are given as one figure. Both the gel concentrate and resulting foams were evaluated for their clarity and thickness. The resulting dental foams were evaluated for the time response from their dispensing gel form to their optimum time of foam generation. The compounding procedure follows below:

Step #1. The prescribed amount of ultra violet treated deionized water is charged into a stainless steel, scale mounted batch tank that is equipped with the proper internal heat source. The water is heated to 185-190° F. Once the amount of water is reached and recorded, additional ingredients can be added. The mixer was continuously in operation once the water was over the mixing paddles.

Step #2. The first water soluble ingredient added is the foam stabilizer, e.g., glycerin. The next ingredient to be added is the sweetening agent, sorbitol as a 70% water solution.

Step #3. Once the batch temperature reached 185-190° F., the oil-in-water emulsifier is added with moderate mixing, e.g., oleth-10-phosphate. Once this ingredient is in solution, the oil-in-water acidulated emulsifier is added to the batch, e.g., oleth-3-phosphate. Care must be taken to insure that the batch is completely in solution before proceeding. The oleth-3-phosphate with a pH of 2.5-3.5 is needed to create the micro-emulsion gel structure.

Step #4. With the mixing batch in complete solution, the required amount of surfactant is added to the batch. After being mixed for a period of time, the gel thickener is added, e.g., PEG-150 Pentaerythrityl tetrastearate, with the temperature at 150-160° F.

Step #5. With the addition of micro-emulsion gel stabilizer and thickener, the compounder will see that the batch becomes more viscous and starts to take on its gel consistency. At this time the gel clarifier is added, e.g., special denatured ethyl alcohol, 200 proof. The denaturant could be from a diverse list of flavored oils or approved denaturing ingredients, such as: benzaldehyde, peppermint oil, spearmint oil, wintergreen oil and many others approved by the U.S. Alcohol, Tobacco & Firearms Department.

Step #6. With continued mixing and temperature down to 110-120° F., the desired flavor and colorant are added with continued mixing. The batch is allowed to cool down to room temperature before starting the actual filling of the aerosol components.

Step #7. After 15-30 minutes of mixing, the micro-emulsion batch is sampled to determine the fluoride content and the pH value at 77° F. The sampling is done from the top, middle, and bottom of the liquid batch.

Step #8. Once the fluoride content is verified and approved at 1.23%±10%, the batch is ready for filling and pressurizing at roughly room temperature.

While the general production procedure shown above demonstrates the manufacture of acidulated dental fluoride foam (pH 3.0-4.0), it is desirable to also be able to treat teeth with a neutral fluoride treatment at a pH of 6.5-7.5. There are a great number of people who are ideal candidates for neutral therapeutic fluoride foam treatment. This patient population may have aesthetic dental restorations, or have an intolerance to acid fluorides due to xerostomia, bulimia, radiation and/or chemotherapy and in-office or home dental teeth whitening. A neutral dental fluoride foam is gentle on bridges, crowns and other dental restorations that may be etched or rendered dull looking due to acidulated fluoride treatment.

In order to prepare a neutral (instead of an acidulated) micro-emulsion that becomes a therapeutic fluoride foam, only a few slight ingredient changes are required. Instead of using oleth-10-phosphate, which is an acidulated oil-in-water emulsifier (pH 2.5-3.0), DEA oleth-10-phosphate (pH 7.0) is used. Likewise, instead of using the acid based oleth-3-phosphate, DEA oleth-3-phosphate (pH 6.5-7.0) is used. Both oil-in-water emulsifiers with a neutral pH are suitable ingredients to produce the gel foamer containing the therapeutic fluoride treatment. The balance of the neutral fluoride gel ingredients and its method of production are exactly the same as described above for the acidulated fluoride gel foam of the present invention.

Micro-Emulsion Gels

The following examples of an oil-in-water micro-emulsion gel formulation were prepared prior to being pressurized. In the following examples, the ancillary ingredients such as preservatives, sweetening agents, flavorings, etc., are recorded as “inactive ingredients”.

### Example #2

Using the manufacturing procedure outlined in Example #1, an oil-in-water acidulated micro-emulsion gel was prepared, per the specific formula set forth below:

Micro-Emulsion Gel #1

| Ingredients | % / Wgt. |
|---|---|
| Deionized Water | 48.8 |
| Sodium lauryl sarcosinate (30% Sol.) | 20.0 |
| Oleth-10-Phosphate | 10.0 |
| Oleth-3-Phosphate | 5.0 |
| Phosphoric Acid (10% Sol.) | 6.0 |
| SDAlcohol, 38B, 200 proof | 5.0 |
| Sodium Fluoride, N.F. | 3.0 |
| Inactives | 2.2 |
| Total: | 100.0 |

Gel #1 resulted in a clear gel that was flavored with a bubble gum taste and colored in preparation for pressurization. It must be noted that in this formulation that the 20% anionic surfactant solution resulted in only a 6.0% concentration based on its active ingredient. Although the high concentration of oil-in-water acidulated emulsifiers adds to the cost of the gel, they did result in a clear micro-emulsion gel being formed.

### Example #3

Micro-Gel #1 Pressurized:

Micro-Gel #1 was pressurized inside the bag-in-can with a 50:50 blend of A17: Isopentane. The propellant outside of the bag was a 4% fill of isobutane (A-31). The unit delivered a good red gel ribbon of product that quickly became a pinkish-white stable foam.

### Example #4

Micro-Emulsion Gel #2:

| Ingredients | %/Wgt. |
|---|---|
| Deionized Water | 64.8 |
| Nonyl phenol ethoxylate, 9.0 mols, E.O. | 6.0 |
| Oleth-10-Phosphate | 8.0 |
| Oleth-3-Phosphate | 5.0 |
| Phosphoric Acid (10% Sol) | 6.0 |
| SD Alcohol 38B, 200 proof | 5.0 |
| Sodium Fluoride, NF | 3.0 |
| Inactives | 2.2 |
| Total: | 100.0 |

In the example the nonionic surfactant was continued at the 6.0% active level. However, the total percentage of oil-in-water hydrophobic emulsifiers was lowered to a total of 13.0% overall. Although the micro-emulsion gels were successfully produced, the taste was not as good as the other micro-emulsions, even though sodium saccharin and various flavoring agents were used.

### Example #5

Micro-Gel #2 Pressurized:

Micro-emulsion gel #2 was pressurized within the bag with a 3% propellant blend of Isobutane: Isopentane 15%:85% by wt. While the gel was flavored with a bubble gum flavorant, it was not colored. The unit dispensed an opaque gel that could not foam to any degree. It was believed that the isobutane had to be increased.

Since the therapeutic fluoride gel and the foam generated subsequently must remain in the mouth and in contact with its teeth for about four (4) minutes, the product taste is a major concern. From a commercial standpoint, in order to be widely used and accepted, the gel and its foam has to taste good. With these concerns, we started to look at the ingredients that would contribute to the bad, sour taste of the micro-emulsion gels disclosed above. We determined by taste that the surfactants, together with the oil-in-water acidulated emulsifiers, were the primary sour tasting ingredients.

Thereafter, we discovered that we could produce a very stable clear, micro-emulsion gel by drastically reduced oil-in-water acidulated emulsifiers. While we had used as much as 10% oleth-10-phosphate together with 5.0% oleth-3-phosphate previously, we found that we could successfully use much less. We discovered that a combination of 4.0% and 2.0% of the acidulated oil-in-water emulsifiers could be successfully used in combination with several new ingredients. The gels produced were clear, stable and could be pressurized without any problems.

Since, our prime goal was to produce a micro-emulsion gel, that then produced by itself a therapeutic fluoride foam for dental caries prevention, we knew that the foam taste had to be acceptable for all ages. In order to help mask the taste of the surfactant and the dual oil-in-water emulsifiers, glycerin was employed and found to be a good foam stabilizer. While glycerin by itself has no taste, it leaves a smooth, silky sensation in the mouth. To further promote a good tasting product, a 70% water solution of sorbitol, which has a pronounced sweet taste was used. The sorbitol and the sodium saccharin worked well together to promote a good tasting gel and fluoride foam. The other ingredient we found to be helpful was PEG-150 Pentaerythrityl tetrastearate, know in trade as Crothix, (Croda Inc., NJ.). We immediately found that Crothix was very efficient micro-emulsion stabilizer and thickener. While still looking to achieve a better tasting product the concentration of the active surfactants were reduced from 6.0% to 1.5% as shown in the following micro-emulsion gel formulations shown below:

### Example #6

Micro-Emulsion Gel #3:

| Ingredients | %/Wgt. |
|---|---|
| Deionized Water | 58.8 |
| Glycerin, USP | 15.0 |
| Sorbitol, 70% Sol. | 6.0 |
| Oleth-10-Phosphate | 4.0 |
| Oleth-3-Phosphate | 2.0 |
| Sodium lauryl sarcosinate, 30% Sol. | 5.0 |
| PEG-150 Penta | 1.0 |
| Sodium Fluoride, NF | 3.0 |
| SD Alcohol 38B, 200 proof | 3.0 |
| Inactives | 2.2 |
| Total: | 100.0 |

Micro-Emulsion Gel #3 gelled extremely well by going into complete solution at 180-185° F. With continued mixing, the product became clear with a stable gel formation. We did notice that upon the addition of the anionic surfactant that the solution seemed to clear. The gel taste was improved but still could be a little better. With this in mind, the glycerin to sorbitol ratio was modified in the next formulation.

Micro-Emulsion Gel #3 Pressurized:

Micro-emulsion Gel #3 was pressurized inside the laminated bag with 2% propellant blend of Isobutane (A-31): Isopentane (25% by wgt.:75% by wgt.). The propellant between the outer bag wall and the can was 4% A-46 (Isobutane:Propane 84.5%:15.5% by wgt.), exerting 46 psig on the outer bag wall. Gel #3 dispensed as a clear gel ribbon that quickly foamed in place. As noted above, we found that the taste was marginal but acceptable, yet we wanted a better tasting product.

### Example #8

Micro-Emulsion Gel #4:

| Ingredients | %/Wgt. |
|---|---|
| Deionized Water | 62.0 |
| Glycerin, USP | 15.0 |
| Sorbitol, 70% Sol. | 6.0 |
| Oleth-10-Phosphate | 4.0 |
| Oleth-3-Phosphate | 2.0 |
| Nonyl phenol ethoxylate, 9.0 mols, E.O. | 1.5 |
| SD Alcohol 38B, 200 proof | 3.0 |
| PEG-150 Penta | 1.3 |
| Sodium Fluoride, NF | 3.0 |
| Inactives | 2.2 |
| Total: | 100.0 |

Micro-Emulsion Gel #4 also went into complete solution with batch temperature being held at 185-190° F. for the initial five ingredients. As the batch temperature was reduced, the remaining ingredients were added with continued mixing. This formulation was flavored with a grape flavoring and colored purple.

### Example #9

Micro-Emulsion Gel #4 Pressurized:

Gel #4 was pressurized as described above with the same 2% blend of A-31/Isopentane (25%/75% by weight) inside the bag-in-can. The noticeable difference from gel #3 above was the dispensing of a purple gel ribbon that developed into a foam after about 40-45 seconds. Since PEG-150 Penta was increased to 1.3% for first time, we could see that the gel structure was thicker, thereby preventing the enclosed propellant blend breaking through to the atmosphere to form the dental therapeutic foam. The dental professional could line the dental trays with a ribbon of micro-emulsion gel and by the time the unit was placed in the patient's mouth, the foam generated would engulf the teeth for a therapeutic fluoride treatment against dental caries.

### Example #10

Micro-Emulsion Gel #5:

| Ingredients | %/Wgt. |
|---|---|
| Deionized Water | 59.3 |
| Glycerin, USP | 15.0 |
| Sorbitol, 70% Sol. | 5.0 |
| DEA Oleth-10-Phosphate | 4.0 |
| DEA Oleth-3-Phosphate | 2.0 |
| Cocoamidopropyl betaine (30% Sol.) | 5.0 |
| SD Alcohol 38B, 200 proof | 3.0 |
| PEG-150 Penta | 1.5 |
| Sodium Fluoride, NF | 3.0 |
| Inactives | 2.2 |
| Total: | 100.0 |

This formulation was flavored with a spearmint taste and colored green. In this example we wanted to formulate a neutral gel that would become a neutral dental therapeutic foam containing the essential fluoride mediant. We substituted the neutral DEA oil-in-water emulsifiers in place of the acidulated oil-in-water emulsifiers. We also wanted to verify the acceptability of an amphoteric surfactant such as cocoamidopropyl betaine in the present invention. We also increased the PEG-150 Penta-stabilizer/thickener for a thicker gel.

### Example #11

Micro-Emulsion Gel #5 Pressurized:

This gel #5 proved to us that the PEG-150 Penta (Crothix) retarded the formation of a stable foam. The gel dispensed as a crystalline green ribbon, but did not foam for 2.5 minutes.

### Example #12

Micro-Emulsion Gel #6:

| Ingredients | %/Wgt. |
|---|---|
| Deionized Water | 60.8 |
| Glycerin, USP | 10.0 |
| Sorbitol, 70% Sol. | 8.0 |
| Oleth-10-Phosphate | 4.0 |
| Oleth-3-Phosphate | 2.0 |
| SD Alcohol 38B, 200 proof | 3.0 |
| Sodium lauryl sarcosinate, 30% Sol. | 5.0 |
| Sodium Fluoride, NF | 3.0 |
| PEG-150 Penta | 1.0 |
| Inactives | 2.2 |
| Total: | 100.0 |

Micro-Emulsion Gel #6 was designed to produce a product with better taste qualities and the glycerin was reduced from previous gel formulations while the sorbitol was increased. Tasting the gel prior to pressurizing told us that the taste improved greatly. By following the method of batching shown above, we could produce a good tasty gel ready for pressurization. To be accepted as a product we used a bubble gum flavor with a water soluble red, food dye solution.

### Example #13

Micro-Emulsion Gel #6 Pressurized:

The aerosol actuator dispensed easily as a clear gel ribbon that almost immediately turned into a pink, hydrophobic foam. We were gratified to see the hydrophobic foam that did not easily dissolve in the presence of moisture. We now know, that a small increased of PEG-150 Penta would have delayed the formation of the pink foam. The concentrations of both glycerin and sorbitol here did help the taste to be right on target.

### Example #14

Micro-Emulsion Gel #7:

| Ingredients | %/Wgt. |
|---|---|
| Deionized Water | 59.5 |
| Glycerin, USP | 12.0 |
| Sorbitol, 70% Sol. | 8.0 |
| Oleth-10-Phosphate | 4.0 |
| Oleth-3-Phosphate | 2.0 |
| SD Alcohol 38B, 200 proof | 3.0 |
| Sodium lauryl sarcosinate, 30% Sol. | 5.0 |
| Sodium Fluoride, NF | 3.0 |
| Crothix | 1.3 |
| Flavoring | 1.5 |
| Preservative | 0.2 |
| Saccharin | 0.5 |
| Total: | 100.0 |

### Example #15

Micro-Emulsion Gel #7 Pressurized:

Micro-emulsion Gel #7 was pressurized inside a laminated bag following the 5 step procedure described above, with 3% by weight of a propellant blend about equal amounts of isobutane (A-17) and isopentane. The bag was then pressurized in the can with about 4% by weight A46 propellant.

### Example #16

Micro-Emulsion Gel #8:

| Ingredients | %/Wgt. |
|---|---|
| Deionized Water | 62.1 |
| Glycerin, USP | 15.0 |
| Sorbitol, 70% Sol. | 5.0 |
| DEA Oleth-10 (neutral) | 4.0 |
| DEA Oleth-3 (neutral) | 2.0 |
| Cocoamidopropyl betaine (30% Sol.) | 5.0 |
| SD Alcohol 38B, 200 proof | 3.0 |
| PEG-150 Penta | 1.5 |
| Sodium Fluoride, NF | 0.2 |
| Inactives | 2.2 |
| Total: | 100.0 |

### Example #17

Micro-Emulsion Gel #8 Pressurized:

Micro-emulsion Gel #8 was pressurized inside a laminated bag following the same procedure as that used for Gel #7.

We claim:

1. A method of treating teeth with fluoride, comprising:
a) dispensing a self-foaming fluoride-containing gel from an aerosol container into the trough of a dental tray;
b) placing the trough of the dental tray containing the self-foaming fluoride containing gel into engagement with the teeth to be treated, and
c) thereafter, allowing the self-foaming gel to transform into a foam while said dental tray is in engagement with the teeth.

2. The method of claim 1 wherein the fluoride-containing gel comprises:
a) a water soluble fluoride component present in an amount sufficient to provide from about 0.5 to about 10.0% by weight available fluoride;
b) from about 2.0 to about 30.0% by weight of an oil-in-water emulsifier;
c) from about 0.5 to about 30.0% by weight of a surfactant;
d) from about 0.5 to about 5.0% by weight of a micro-emulsion thickener;
and optionally
e) from about 0.5 to about 10.0% by weight of a gel clarifying agent.

3. The method of claim 1 wherein the fluoride-containing gel contains from about 0.5 to about 5.0% available fluoride.

4. The method of claim 3 wherein the fluoride-containing gel contains from about 1.0 to about 3.0% available fluoride.

5. The method of claim 1, wherein said water soluble fluoride component is selected from the group consisting of sodium fluoride, sodium monofluorophosphate, stannous fluoride, fluoroalkylphosphate salts, quaternary ammonium fluorides and mixtures thereof.

6. The method of claim 5, wherein said water soluble fluoride component is sodium fluoride.

7. A foamable dental fluoride gel composition, comprising:
a) from about 0.5 to about 10.0% by weight of a water soluble fluoride component;
b) from about 2.0 to about 30.0% by weight of an oil-in-water emulsifier;
c) from about 0.5 to about 30.0% by weight of a surfactant; and
d) from about 0.5 to about 5.0% by weight of a micro-emulsion thickener;

8. The foamable dental fluoride gel of claim 7, wherein said water soluble fluoride component is selected from the group consisting of sodium fluoride, sodium monofluoro-phosphate, stannous fluoride, fluoroalkylphosphate salts, quaternary ammonium fluorides and mixtures thereof.

9. The foamable dental fluoride gel composition of claim 8, wherein said sodium fluoride is present in an amount of from about 1.0 to about 22.2% by weight.

10. The foamable dental fluoride gel composition of claim 9, wherein said sodium fluoride is present in an amount from about 1.0 to about 10.0% by weight.

11. The foamable dental fluoride gel composition of claim 10, wherein said sodium fluoride is present in an amount from about 2.0 to about 6.0% by weight.

12. The foamable dental fluoride gel composition of claim 7, wherein said oil-in-water emulsifier is a blend of a first and a second emulsifier.

13. The foamable dental fluoride gel composition of claim 12, wherein the ratio of said first emulsifier to said second emulsifier is about 2:1.

14. The foamable dental fluoride gel composition of claim 12, wherein said first emulsifier present in an amount of from about 1.0 to about 20.0% by weight.

15. The foamable dental fluoride gel composition of claim 14, wherein said first emulsifier is present in an amount of from about 1.0 to about 10.0% by weight.

16. The foamable dental fluoride gel composition of claim 7, wherein said emulsifier is selected from the group consisting of cetyl phosphate, stearic acid, PPG-5, ceth phosphate, cetearyl alcohol, dicetyl phosphate, ceteth-10 phosphate, polyoxyethylene (10) oleyl alcohol phosphate, polyoxyethylene (3) oleyl alcohol phosphate, cetostearyl alcohol, stearyl alcohol, oleyl alcohol, behenic acid, cetyl phosphate, stearic acid, fatty alcohols with linear carbon chains, phosphated cetyl ether and mixtures thereof.

17. The foamable dental fluoride gel composition of claim 12, wherein said first is polyoxyethylene (10) oleyl alcohol phosphate.

18. The foamable dental fluoride gel composition of claim 12, wherein said second emulsifier is present in an amount from about 1.0 to about 10.0% by weight.

19. The foamable dental fluoride gel composition of claim 18, wherein said second emulsifier is present in an amount from about 1.0 to about 6.0% by weight.

20. The foamable dental fluoride gel composition of claim 12, wherein said second emulsifier is polyoxyethylene (3) oleyl alcohol phosphate.

21. The foamable dental fluoride gel composition of claim 12, wherein said first emulsifier is polyoxyethylene (10) oleyl alcohol phosphate and said second emulsifier is polyoxyethylene (3) oleyl alcohol phosphate.

22. The foamable dental fluoride gel composition of claim 12, wherein said first emulsifier is DEA oleth-10-phosphate and said second emulsifier is DEA oleth-3-phosphate.

23. The foamable dental fluoride gel composition of claim 7, wherein said surfactant is present in an amount from about 1.0% to about 10.0% by weight.

24. The foamable dental fluoride gel composition of claim 7, wherein said surfactant is selected from sodium lauryl ether sulfate, ammonium lauryl ether sulfate, sodium lauryl sarcosinate, sodium-N-methyl-N-oleyl-taurate, Disodium N-oleyl sulfosuccinate, Nonyl-phenol ethoxylate, myristyl-cetyl dimethyl oxide, lauric acid diethanolamine, Cocoamidopropyl betaine, lauryl amine oxide, coco imidazoline monocarboxylate, Cocoamphocarboxyl glycinate, Bis (2-hydroxy-ethyl) cocoaoxide, cetyl dimethyl benzyl ammonium chloride, Dimethyl aminopropylamine and mixtures thereof.

25. The foamable dental fluoride gel composition of claim 7, wherein said micro-emulsion thickener is present in an amount from about 1.0 to about 3.0% by weight.

26. The foamable dental fluoride gel composition of claim 7, wherein said micro-emulsion thickener is selected from the group consisting of cetyl alcohol, sodium monostearate, PEG-150 Pentaerythrityl tetrastearate and mixtures thereof.

27. The foamable dental fluoride gel composition of claim 26, wherein said micro-emulsion thickener is PEG-150 Pentaerythrityl tetrastearate.

28. The foamable dental fluoride gel composition of claim 7, further comprising from about 0.5 to about 10.0% by weight of a gel clarifying agent.

29. The foamable dental fluoride gel composition of claim 28, wherein said clarifying agent is present in an amount from about 1.0 to about 6.0% by weight.

30. The foamable dental fluoride gel composition of claim 28, wherein said clarifying agent is denatured ethyl alcohol.

31. An aerosol container for delivering a self foaming dental fluoride gel, comprising:
i) a barrier bag within said aerosol container comprising
a) a water soluble fluoride component present in an amount sufficient to provide from about 0.5 to about 10.0% by weight available fluoride;
b) from about 2.0 to about 30.0% by weight of an oil-in-water emulsifier;
c) from about 0.5 to about 30.0% by weight of a surfactant;
d) from about 0.5 to about 5.0% by weight of a micro-emulsion thickener;
f) from 0 to about 10.0% by weight of a gel clarifying agent; and
g) from about 2.0 to about 8.0% by weight of a first propellant having a vapor pressure of from about 10 to about 46 psig; and
ii) a pressurized region within said aerosol container and surrounding said barrier bag, said pressurized region containing a second propellant having a vapor pressure of from about 14 to about 20 psig greater than vapor pressure of said first propellant.

32. The aerosol container of claim 31, wherein said first propellant is a blend of about equal parts n-butane and isopentane.

33. The aerosol container of claim 31, wherein said first propellant is a blend of about 20% by weight isobutane and 80% by weight isopentane.

34. The aerosol container of claim 31, wherein said first propellant is present in an amount of about 3% by weight.

35. The aerosol container of claim 31, wherein said second propellant is a mixture of isobutane and propane, and said mixture has a vapor pressure of about 46 psig.

36. The aerosol container of claim 31, wherein said second propellant is present in an amount of about 4% by weight.
